(19) United States
(12) Patent Application Publication
(10) Pub. No.: US 2005/0065438 A1
(43) Pub. Date: Mar. 24, 2005

(54) System and method of capturing and managing information during a medical diagnostic imaging procedure

(76) Inventor: Landon C.G. Miller, Tuscaloosa, AL (US)

Correspondence Address: GIFFORD, KRASS, GROH, SPRINKLE;ANDERSON & CITKOWSKI, PC, 280 N OLD WOODARD AVE, SUITE 400, BIRMINGHAM, MI, 48009, US

(21) Appl. No.: 10/936,035

(22) Filed: Sep. 8, 2004

Related U.S. Application Data
(60) Provisional application No. 60/501,620, filed on Sep. 8, 2003.

(51) Int. Cl. A61B 8/00

(52) U.S. Cl. 600/437

(57) ABSTRACT

A system and method of capturing and managing the medical information obtained during an imaging procedure is provided. The system includes a sonogram imaging device operated by a technician, and a technician computer system in communication with the imaging device via a communications network. A computer server communicates with the technician computer system, and includes a medical diagnostic imaging software program. A healthcare provider computer system is operatively in communication with the computer server via the communications network. The method includes the steps of conducting the imaging procedure and building a study from the generated image and captured impression of the real-time observations of the generated image and measurement data by the technician. The study is stored in the database associated with a computer server and is accessible by a healthcare professional using a healthcare provider computer system for preparing a medical diagnosis.

### RELATED APPLICATION

This application claims priority of U.S. Provisional Patent Application Ser. No. 60/501,620 filed Sep. 8, 2003, which is incorporated herein by reference.

### BACKGROUND OF THE INVENTION

I. Field of the Invention

The present invention relates generally to medical procedures and more particularly to a system and method of capturing and managing health information obtained during a medical diagnostic imaging procedure.

II. Description of the Prior Art

Advances in biomedical engineering have resulted in a proliferation of medical devices used for diagnostic purposes. In particular, various medical diagnostic imaging devices are well known in the art, such as magnetic resonance imaging (MRI), ultrasound, endoscopy, or computed tomography (CT). These imaging devices are usually standalone systems and are usually operated by highly trained technicians.

Medical diagnostic imaging devices typically include a processor for processing the information generated during a diagnostic test procedure. For example, an ultrasound machine is known to include software for capturing data while controlling hardware systems related to a specific ultrasound procedure. These previously known systems include, but are not limited to, encapsulated hardware and software combinations. There are a number of available centralized systems known as PACS, or picture archiving control systems, which act as collectors of the images from individual standalone systems, also called modalities, such as a sonogram machine.

Despite the extensive diagnostic capabilities of these imaging devices, however, there is no formal system and method available for managing the collection of the data and measurements derived during a test procedure, as well as the technician's impression. The collected data and technician's impressions and observations in real time of the procedure is extremely useful for diagnostic purposes.

While these imaging devices work well, a primary disadvantage is that the physician reviewing the data generated during a test procedure is merely reviewing discrete images or information obtained during a dynamic testing procedure in order to make a diagnosis. The existing test devices only provide the physician, such as a radiologist, with discrete data and images. Unfortunately, in the case of imaging procedures such as sonogram or ultrasound imaging, the discrete data may not be sufficient. Sonogram imaging and image analysis is an experience-based process, or “art.” An accomplished sonogram technician, or sonographer, is an important part of the diagnostic and interventional radiological team. The experienced sonographer's impression of the data and observation of real-time images as a result of their presence during the test procedure is an important piece of information needed by the radiologist in order to accurately interpret such studies.

Additionally, there may be a significant amount of detailed data and measurements associated with an imaging procedure, such as a sonogram study. Ultrasound images are developed using low energy sonar or sound waves and are indistinct as compared to other radiologist diagnostic images. An experienced sonographer can understand these indistinct images and perform a wide variety of data gathering and measurements processes, as well as generally describe the medical situation, or their impression of the subject patient, in real time.

For all these reasons, written forms were developed in order to capture the data and the technician's impressions and observations in a format useful to the radiologist in analyzing and interpreting the information, and then preparing a report with the diagnosis. This information may then be utilized in a medical intervention to perform the appropriate medical procedures. For example, the radiologist report is used in performing a biopsy on potentially cancerous tissue. In the past, these forms were cumbersome, expensive, and created opportunities for transcription error. Also, the radiologist would have to dictate long sequences of data and measurements into the form. This situation also creates an opportunity for error, and generally slows down the overall diagnostic process and intervention. Another disadvantage is that the forms may by incomplete, or restrictive due to space limitations.

Thus, there is a need in the art for a computer-based system and method for capturing and managing a test operator's impression and their supporting measurements during a test procedure, and transmitting the data to a remotely located computer system in pre-formatted text format. The data is available for diagnostic interpretation by a physician, and may be available for further interventional medical procedures.

### SUMMARY OF THE PRESENT INVENTION

A system and method of capturing and managing the data obtained during a medical diagnostic imaging procedure is provided. The system includes a sonogram imaging device operated by the technician and a technician computer system operatively in communication with the imaging device via a communications network. A computer server is operatively in communication with said technician computer system, and includes a medical diagnostic imaging software program for capturing and managing information obtained by the technician during the medical diagnostic imaging procedure. A healthcare provider computer system is operatively in communication with the computer server via the communications network.

The method includes the steps of conducting the imaging procedure on the patient and building a study from the generated image and capturing an impression of the medical diagnostic imaging procedure based on real-time observations of the generated image and measurement data by the technician, and including the captured impression, in the study. The study is stored in the database associated with a computer server and is accessible by a healthcare professional using a healthcare provider computer system for preparing a medical diagnosis.

One advantage of the present invention is that a system and method of capturing and managing data obtaining during a medical diagnostic imaging procedure is provided that improves the accuracy of diagnostic interpretation of the data. Another advantage of the present invention is that a system and method is provided that links information technology with medical diagnostic technology. Still another advantage of the present invention is that a system and method is provided that reduces the amount of time it takes to evaluate the data, since the data is provided in a consistent, organized manner. A further advantage of the present invention is that a system and method is provided that efficiently manages the diagnostic data so that associated costs are reduced. Still a further advantage of the present invention is that a system and method is provided that improves diagnostic accuracy and intervention through enhanced information. Still yet a further advantage of the present invention is that a system and method is provided for capturing the real-time impressions and observations of the test administrator.

Other features and advantages of the present invention will be readily understood as the same become better understood after reading the subsequent description when considered in connection with the accompanying drawings.

### BRIEF DESCRIPTION OF THE DRAWING

FIG. 1 is a diagrammatic view illustrating a system of capturing and managing the data obtained during a medical diagnostic imaging procedure, according to the present invention;

FIG. 2 is a flowchart illustrating a method of managing the data obtained during a medical diagnostic imaging procedure using the system of FIG. 1, according to the present invention; and

FIGS. 3A-3E are diagrammatic views illustrating the application of the system of FIG. 1 and method of FIG. 2, according to the present invention.

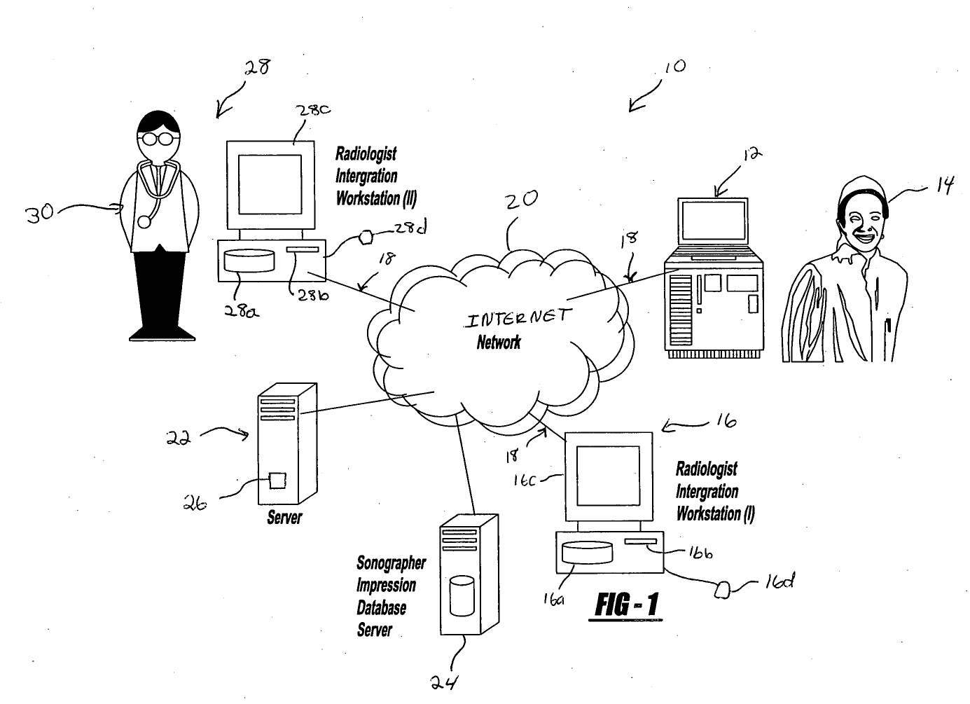

DETAILED DESCRIPTION OF PREFERRED EMBODIMENT(S) Referring to FIG. 1, a system 10 is disclosed for capturing and managing data obtained during a medical diagnostic imaging procedure. It should be appreciated that in this example the imaging procedure is a sonogram performed using a sonographic device by a technician, referred to in the art as a sonographer. The system and method captures the impressions or observations of the ultrasound medical sonographer and the data upon which such impression is based, in real time, and stores the information on a computer system having a data storage means containing a database.

The system 10 includes an imaging device 12 operated by a technician 14. In this example, the imaging device 12 is an ultrasound imaging device that includes an ultrasonic transducer, a processor, a transmitter and a receiver. The ultrasonic transducer generates an ultrasonic signal that is transmitted to the processor. The processor collects the data and constructs a real-time image from the collected data. It should be appreciated that the image may be reconstructed in a multidimensional form, such as two-dimensional or three-dimensional. For example, the ultrasonic image of the uterus of a pregnant woman allows for the visualization of the fetus, in order to gain information concerning the fetus.

The system 10 includes a technician computer 16 having a memory 16a, a processor 16b, a display screen 16c and an input device 16d. In this example, the technician computer 16 is separate from the imaging device 12; however, it is contemplated that they could be integral and one. Various types of user input devices are considered, such as a mouse and a point and click technique, or voice recognition, or a keyboard or the like. A combination of input devices could be utilized. The technician computer 16 receives an output signal generated by the imaging device, and processes the signal in a manner to be described. It should be appreciated that the technician computer 16 may be a personal computer, a portable computing device, such as a notebook computer, a handheld computer, a palm-held organizer, a web-enabled cellular phone, or the like that provides computing ability, information storage, and information retrieval. The technician computer is operatively in communication with the imaging device or the rest of the system 10 via a communications network 18.

The communications network 18 can include various types of communication means. One example of a communication means is a wire (e.g. phone line or cable line or the like) operatively connecting the components. Alternatively, the communication means 18 is a wireless link. One example of a wireless link is a universal shortwave connectivity protocol referred to as Bluetooth. Another example of a wireless link is a memory module, also known as a memory stick or memory card. Still another example of a wireless link is satellite communication. Other types of telecommunication links are contemplated. It is contemplated that more than one type of communication means can be utilized within the communications network 18.

The communication network 18 may be interconnected via the communication means through an internet 20. An internet, such as the Internet, includes providers, such as Internet Access Providers (IAPs), Internet Service Providers (ISPs), Network Service Providers (NSPs) and routers that provide wired and wireless digital telecommunication throughout the world. In this example, the communication is provided using a TCP/IP networking protocol. It should be appreciated that the system components may access the Internet directly, or they may be operatively connected to a Local Area Network (LAN) over which information is transmitted to other computers on the same LAN, or to computers on other LANs through a localized intranet.

The system also includes a website server 22 that interactively transfers information to a user through the user's computer via the communication means 18, and the Internet 20. The website server 22 is a computer system operatively connected to the Internet via the telecommunications link 18, as previously described. The website server 22 provides for interactive communication between the host of the website and a visitor to the website. The communication is facilitated by a series of web browser screens, also referred to as pages, and displayed on the display device. The first page is referred to as a home page. When a user visits a particular website, the user is served a page displayed on the display device and referred to as a “home page”. The user may interact with the website pages via the user input device by making a selection or request.

The website server 22 is operatively in communication with a data storage means 24, via the communications network 18. The data storage means is conventionally known in the art, and may include both randomly accessed memory as well as persistent data storage, such as a hard drive. The data storage means 24 may also include software for managing the data storage device. The information is retrieved and displayed in the form of web pages. The server 22 stores and displays the information in various ways, either by itself or in combination with other previously stored information pertinent to a particular patient. The server 22 utilizes various software programs to process and present the information.

The website server 22 further includes a medical diagnostic imaging procedure software program stored on the web server. The medical diagnostic imaging procedure software program may include an access control means 26 for controlling access to data records. Various types of access control means 26 are contemplated, such as a combination of security certificates or password access control or the like. The medical diagnostic imaging procedure software program may include software for implementing other features, such as administrative support, including user naming and password control, alternate image viewing support, imaging device work list date range selection. The medical diagnostic imaging procedure software program may also include a feature for quickly building a list containing elements such as common comments useful in quickly selecting and entering comments into the system regarding the medical diagnostic imaging procedure. The medical diagnostic imaging procedure software program may also include a feature for making a modification to a record, such as to the patient's demographic information. Other software features may provide for reporting, auditing, and data purge capabilities.

The system website server 22 manages the data stored within the data storage database 24 pertaining to one or more patient ultrasound studies. Furthermore, the server 22 may be preprogrammed with security access means to limit access to only the data pertaining to a particular patient, or to those individuals associated with that particular patient's medical information. Preferably, the system 10 assigns a password to a technician 14 or other medical personnel, to control privacy of a particular patient's medical diagnostic imaging procedure study.

The system 10 further includes a healthcare provider computer system 28. The healthcare provider computer system 28 includes a memory 28a, a processor 28b, a display screen 28c and an input mechanism 28d. It should be appreciated that that the healthcare provider computer may be a personal computer, a portable computing device, such as a notebook computer, a handheld computer, a palm-held organizer, a web-enabled cellular phone, or the like that provides computing ability, information storage, and information retrieval. The healthcare provider computer 28 is operatively in communication with the rest of the system 10 via the communication network, as previously described. The healthcare provider 30 can be a physician, or other healthcare professional. Advantageously, the physician can study the data or images generated during a particular imaging study, interpret the results and make a diagnosis. In addition, the results of the study may be utilized in a medical intervention in real time. The system may include various system navigation aids to facilitate usage of this program.

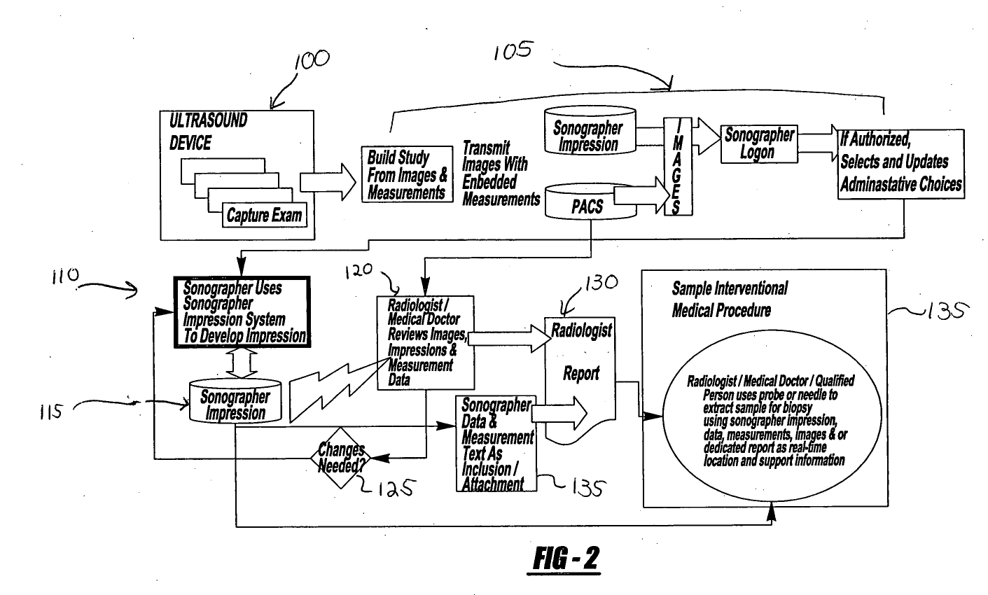

Referring to FIG. 2, a method for capturing and managing the data obtained during a medical imaging procedure is provided using the system of FIG. 1. The method begins in block 100, and the imaging device 12 is utilized by the technician during a medical diagnostic imaging procedure 14 to generate an image of the patient. The imaging device 12 transmits a signal via the communications network to the technician computer system for processing. The methodology advances to block 105.

In block 105, the medical diagnostic imaging procedure software program is utilized to build a study from the transmitted information, including generated images and measurements. The technician 14, such as the sonographer of this example, selects a diagnostic procedure from a predetermined list of medical imaging diagnostic procedures. The technician is provided with a web page containing data prompts for the selected medical procedure, and the technician inputs data into the technician computer system 16 during the medical procedure. For example, the technician 14 may utilize the medical diagnostic imaging procedure software program on the server 22 to access a web page and select a main menu. The technician 14 may select to enter information gathered during the procedure, such as measurement data, including length, velocity, width, and depth. These measurements are preferably captured using a set of predetermined electronic data capture web browser screens, to be described, which prompt the technician 14 to capture or record the data and the technician's impressions or observations in real time during the imaging procedure. It should be appreciated that the images and measurements are discrete pieces of information, while the technician's impression is based upon continuous observations during the test procedure. The captured information is preferably stored in the database system 24, as previously described. The information is available for use by another healthcare professional 30, typically a radiologist or other medical doctor, who has access to the system 10.

It should be appreciated that a combination of these interrelated measurements may be utilized in making a single exam, and one or more exams may define a study.

In this example, the system 10 notifies the technician 14 of potential patients. For example, a DICOM compatible system worklist is utilized. An optional DICOM compatible image viewer may be provided if the system 10 does not have a PACS system. The images may be viewed, such as using the PACS 10 server or, alternatively, on the technician computer system 16.

On another web browser screen, the technician 14 may enter the patient demographic information, as well as all relevant measurements for the selected test procedure. At the completion of the entering of the data and measurements into the web browser based information capture program, the methodology advances to block 110.

In block 110, the technician captures his impression of the selected procedure using the diagnostic imaging procedure software. The technician's impression may further analyze the images, data and measurements generated during the exam. The technician may utilize the detailed features and capabilities of the system to develop and finalize their impression. The methodology advances to block 115. In block 115, the study, including test procedure data and technician impression, is transmitted to the website server and stored in the associated database.

The methodology advances to block 120, and a healthcare provider 30, such as a radiologist or other qualified medical person, retrieves the study, which includes the data and technician's impression, from the database and evaluates the study in order to interpret the medical diagnostic imaging study. This interpretation is useful in forming a medical diagnosis concerning the patient.

Advantageously, the medical diagnostic imaging procedure software program includes a means for alerting the healthcare provider that a new study is available for diagnostic purposes. For example, the healthcare provider 30 may use a means such as an unread impression list to select a study, or a hyperlink generated by the system to select a study. The healthcare provider 30 accesses and reviews the data generated during the study. The healthcare provider 30 also utilizes the real-time impressions and observations of the technician 14 captured during the test procedure in making a diagnosis.

It should be appreciated that the healthcare provider may periodically review, in a web-based program provided by the medical diagnostic imaging procedure software and server 22, a list of unread or unanalyzed technician's impressions. For example, the radiologist may select a study from the list, and then review the images using the PACS server or alternatively, using a DICOM compatible viewer, along with the detailed data and measurements, and the technician's captured impression from the study. This information is used to prepare a medical interpretation or diagnostic report. The report is stored in the system database and may be available to other medical professionals as needed. The report may be provided as a preformatted text output from the system server. The study may be generated upon the user's selection of the study and clicking on a software submit button for the selected study contained in the web browser based software list program previously described above. The system 10 maintains the records for the patient, including any changes to a data file for the patient.

Optionally, the methodology may advance to decision block 125 and the healthcare provider 30 may decide to consult with the technician 14 concerning the captured impressions of the technician. As a result of this consultation, the medical diagnostic imaging procedure software program may include a feature for revising the captured impression of the technician.

Returning to block 130, the healthcare provider 30 uses the study, including the captured impression of the technician that is based on continuous observation, in interpreting the discrete images and measurement and other data generated during the test procedure. The healthcare provider's interpretation results in a diagnosis. The diagnosis may be memorialized in a report. Preferably, the diagnostic report is also stored on the computer system. It should be appreciated that the technician's report may be included as an attachment to the healthcare provider's report.

The methodology advances to block 135 and the report, the diagnostic report, including the technician's impression, the study images, and other medical information, such laboratory test results, are used in real time by a healthcare professional to perform a medical interventional procedure. Examples of procedures include, but are not limited to, the insertion of a needle into the patient to extract a small bit of matter from the person at a location determined during the sonographer's study and subsequent impression building process. Other medical interventional procedures which use the technician's impression and supporting data and measurements (the study) in real time include direct surgical procedures, radiation treatments, and the like. In the case of interventional procedures, the images and the impression may be used to capture additional images in real time. For example, additional images may be required to precisely insert a needle into a correct location to locate a particular internal mass and take a small sample, or biopsy, for further analysis and testing.

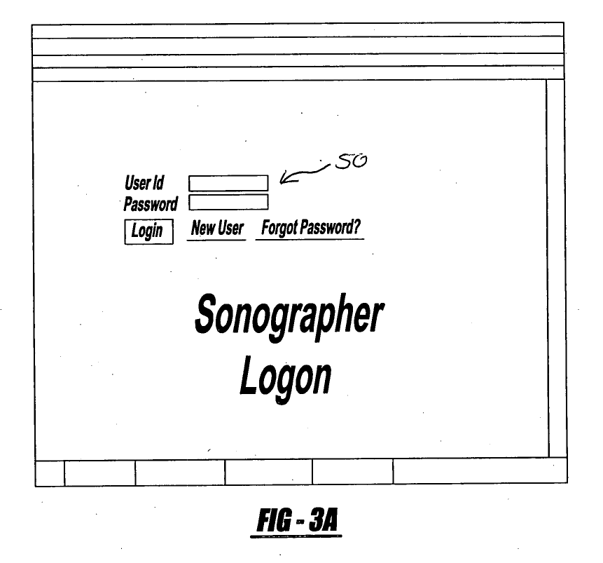

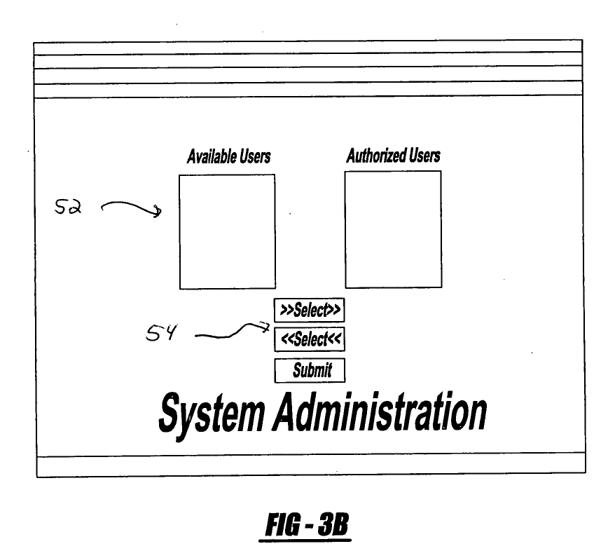

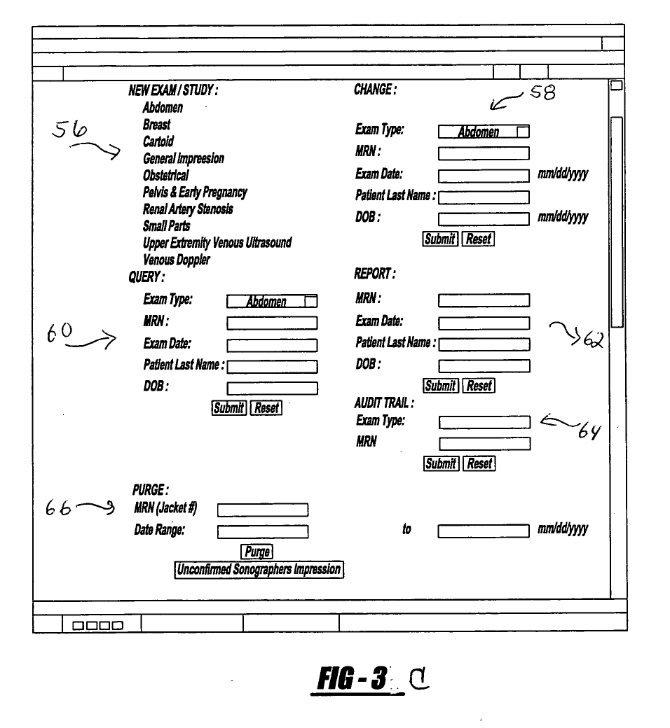

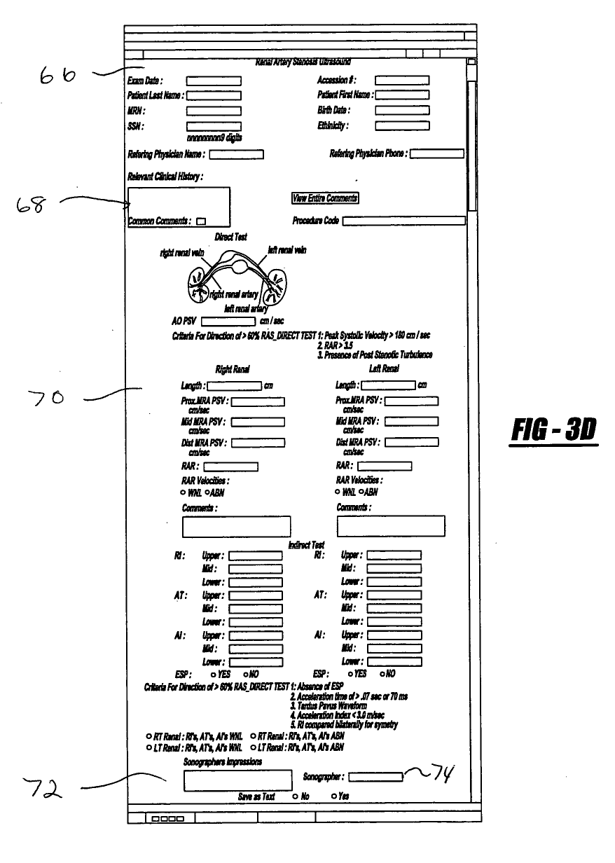

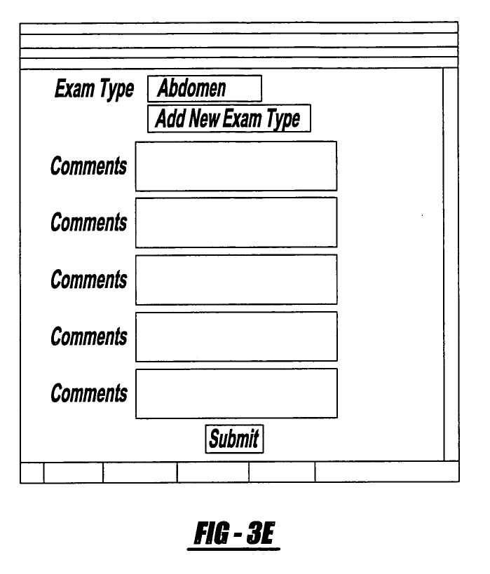

Referring to FIGS. 3A to 3E, an example of the web browser screens provided by the medical diagnostic imaging software program through the methodology of FIG. 2 using the system of FIG. 1 are illustrated. Referring to FIG. 3A, a user, such as a healthcare professional, technician, or sonographer of this example, initiates their use of the system 10 by accessing a web page, referred to as a logon screen, to gain access into the system database 24. The system 10 may control access to the database 24. For example, through the use of an SSL certificate, or special encryption subsystem, or the use of managed passwords as shown at 50.

Referring to FIG. 3A, the system provides a general administrative control subsystem access web browser screen, in which a previously identified and pre-established special operator user prepares and maintains a list of allowable users as shown at 52. For example, a system administrative function may establish appropriate user logon identification. In another function, common comments may be recalled in a dropdown list. Another system administration function may set top level administrative responsibilities via unique logon identification. Other features may include the insertion of a local organization logo for common appearance throughout the system, or entry of local information which will appear in each technician's report.

The screen may also include a list of common comments for a selected imaging procedure, as shown at 54. It should be appreciated that a selected imaging procedure may include common web browser data, measurement and capture screens. Also, a date range for the imaging device work list search, which is used to query the imaging device or the PACS system, may be specified on this screen.

If the user is allowed access into the database 24, the user, such as the technician or healthcare provider, is presented with a main menu web browser screen, as shown in FIG. 3C. The screen may display an alert means indicating that a patient data record stored in the server database is complete. The alert means may further include a feature for notifying said person(s) of any identified incompletions.

The user may utilize the user input device to select an option. One example of an option is a predefined medical procedure, as shown at 56. The system 10 includes a web information capture screen associated with each of the various predefined medical diagnostic imaging procedures. Examples of medical procedures and related information capture screens include, but are not limited to, medical information about the abdomen, the breasts, carotid, general studies or general impression, obstetrical and fetus, the female pelvis and early pregnancy conditions, studies regarding renal artery stenosis, the ultrasound analysis of various small parts of the body, analysis of the upper extremity venous using ultrasound techniques, and analysis of venous Doppler rates and conditions. One example of a category is patient demographics, measurements, data and impression capture screens that support all ultrasound procedure codes. The selections may also include a change control area for first selecting and then changing such data as needed. The selections may further include a general query capability which allows the sonographer user to select and then review the patient studies and their individual exams as shown at 60. The selection menu may further include a reporting function which permits the sonographer to select a patient and then produce preformatted paper printouts of the relevant selected studies, as shown at 62. The selection menu may still further include an audit trail capability which permits the sonographer to display or print a complete history of every change made to a patient study stored in the system database 24, as shown at 64. The selection menu page may further contain a purge option for use in deleting patient records if necessary, as shown at 66.

Referring to FIG. 3D, the user may be presented with a web page containing detailed study information. Each of the information screens for a particular medical procedure contains general areas of information capture and subsequent display. For example, the information capture screen may include a patient demographic section as shown at 66, a general comment section which uses the common comments added during the administrative process to speed information capture completion as shown at 68, and a specific data and measurements capture and analysis section. It should be appreciated that this section may be customized according to the particular medical procedure being performed. The information capture screen may further include a general operator impression capture section, as shown in FIG. 3E. In addition, the information capture screen may also include a means for electronically signing the operator impression as shown at 74.

The system 10 may include various system navigation aids that are known in the art to facilitate the usage of this software program. Also, other screens are contemplated to carry out this program.

The present invention has been described in an illustrative manner. It is to be understood that the terminology which has been used is intended to be in the nature of words of description rather than of limitation. Many modifications and variations of the present invention are possible in light of the above teachings. Therefore, within the scope of the appended claims, the present invention may be practiced other than as specifically described.

1. A system of capturing and managing the medical information obtained during a medical diagnostic imaging procedure performed on a patient comprising:
a sonogram imaging device operated by a technician during a medical diagnostic imaging procedure to construct an image of a patient;
a technician computer system operatively in communication with said imaging device via a communications network, wherein said technician computer system includes a processor, a memory, a display device and an input device;
a computer server operatively in communication with said technician computer system, wherein said computer server includes a memory, a processor and a data storage means containing a database, said computer server being accessible by one or more persons,
a medical diagnostic imaging software program resident in the memory of said computer server for capturing and managing information obtained by the technician during the medical diagnostic imaging procedure, and the technician accesses said medical diagnostic imaging information capture software program using said technician computer system during the medical diagnostic imaging procedure to record discrete data and to record an impression of a continuous observation of the technician of the medical diagnostic imaging procedure, in a patient diagnostic study, and the patient diagnostic study is stored in the server database; and
a healthcare provider computer system operatively in communication with said computer server via said communications network, wherein said healthcare provider computer system includes a memory, a processor, a display device and an input device, and a healthcare provider utilizes said healthcare provider computer system to access the patient diagnostic study stored in the server database, and utilizes the patient diagnostic study to formulate a diagnosis.

2. A system as set forth in claim 1 wherein said medical diagnostic imaging software program provides a web page containing a predetermined list of data entry options for a predetermined medical diagnostic imaging procedure and the technician selects from the list of data entry options to record the medical imaging procure data and the technician's impression of the medical diagnostic imaging procedure.

3. A system as set forth in claim 1 wherein said medical diagnostic imaging software includes an alert means for notifying the healthcare professional of the availability of the medical diagnostic study.

4. A system as set forth in claim 1 wherein said sonogram imaging device is an ultrasound device having an ultrasonic transducer, a processor, a transmitter and a receiver.

5. A system as set forth in claim 1 wherein said communications network includes wired and/or wireless communication over an internet.

6. A system as set forth in claim 5 wherein said internet is the Internet.

7. A system as set forth in claim 5 wherein said internet is an intranet.

8. A method of capturing and managing medical information about a patient obtained during a medical diagnostic imaging procedure, said method comprising the steps of:
generating image and measurement data of the patient by conducting a medical diagnostic imaging procedure on the patient by a technician using an imaging device;
building a study from the generated image and measurement data using a medical diagnostic imaging software program that is resident in the memory of a computer server for capturing and managing the data obtained by the technician during the medical diagnostic imaging procedure, and the technician accesses the medical diagnostic imaging information capture software program using a technician computer system operatively in communication with the computer server via a communications network, and the technician computer system is operatively in communication with the imaging device via a communications network, and the technician computer system includes a processor, a memory, a display device and an input device, and the computer server includes a processor and a data storage means containing the database;
capturing an impression of the medical diagnostic imaging procedure by the technician based on real-time observations of the generated image and measurement data, and including the captured impression in the study;
storing the study in the database associated with a computer server;
accessing the study by a healthcare professional using a healthcare provider computer system operatively in communication with the computer server via the communications network, wherein the healthcare provider computer system includes a memory, a processor, a display device and an input device;
analyzing the study by the healthcare provider and using the study to formulate a medical diagnosis.

9. A method as set forth in claim 8 further comprising the step of notifying the healthcare provider of the availability of a new study stored in the server database.

10. A method as set forth in claim 8 further including the steps of:
consulting with the technician by the healthcare provider regarding the captured impression of the technician.

11. A method as set forth in claim 10 further including the step of updating the captured impression of the technician if determined that the captured impression should be updated after the healthcare provider consults with the technician.

12. A method as set forth in claim 8 further comprising the step of using the study in real time by the healthcare professional to perform a medical interventional procedure.

13. A method as set forth in claim 8 wherein the communications network includes wired and/or wireless communication over an internet.

14. A method as set forth in claim 8 wherein said step of building a study further includes the steps of:
providing the technician a main menu web browser screen on the technician's computer system, wherein the main menu web browser screen includes a menu of diagnostic procedures;
selecting a medical diagnostic procedure by the technician from the menu of diagnostic procedures;
providing the technician with a data entry web browser screen for the selected medical diagnostic procedure on the technician's computer system;
using the data entry web browser screen to enter data and measurements for the selected medical diagnostic procedure by the technician;
providing the technician with an impression entry web browser screen for the selected medical diagnostic procedure on the technician's computer system; and
using the impression entry web browser screen to capture the impression of the technician in observing the selected medical diagnostic procedure in real time.

15. A method of capturing and managing medical information about a patient obtained during a medical diagnostic imaging procedure, said method comprising the steps of:
providing the technician a main menu web browser screen on the technician's computer system, wherein the main menu web browser screen includes a menu of diagnostic procedures;
selecting a medical diagnostic procedure by the technician from the menu of diagnostic procedures;
generating image and measurement data of the patient by conducting a medical diagnostic imaging procedure on the patient by a technician using an imaging device;
building a study from the generated image and measurement data using a medical diagnostic imaging software program that is resident in the memory of a computer server for capturing and managing the data obtained by the technician during the medical diagnostic imaging procedure, and the technician accesses the medical diagnostic imaging information capture software program using a technician computer system operatively in communication with the computer server via a communications network, and the technician computer system is operatively in communication with the imaging device via a communications network, and the technician computer system includes a processor, a memory, a display device and an input device, and the computer server includes an processor and a data storage means containing the database;
providing the technician with a data entry web browser screen for the selected medical diagnostic procedure on the technician's computer system;
using the data entry web browser screen to enter data and measurements for the selected medical diagnostic procedure by the technician;
providing the technician with an impression entry web browser screen for the selected medical diagnostic procedure on the technician's computer system;
capturing an impression of the medical diagnostic imaging procedure by the technician using the impression entry web browser screen, based on real-time observations of the generated image and measurement data, and including the captured impression in the study;
storing the study in the database associated with a computer server;
accessing the study by a healthcare professional using a healthcare provider computer system operatively in communication with the computer server via the communications network, wherein the healthcare provider computer system includes a memory, a processor, a display device and an input device;
analyzing the study by the healthcare provider and using the study to formulate a medical diagnosis.

16. A method as set forth in claim 15 further comprising the step of notifying the healthcare provider of the availability of a new study stored in the server database.

17. A method as set forth in claim 15 further including the step of:
consulting with the technician by the healthcare provider regarding the captured impression of the technician.

18. A method as set forth in claim 15 further including the step of updating the captured impression of the technician if determined that the captured impression should be updated after the healthcare provider consults with the technician.

19. A method as set forth in claim 15 further comprising the step of using the study in real time by the healthcare professional to perform a medical interventional procedure.

20. A method as set forth in claim 15 wherein the communications network includes wired and/or wireless communication over an internet.
